(19) United States
(12) Patent Application Publication
(10) Pub. No.: US 2015/0002647 A1
(43) Pub. Date: Jan. 1, 2015

(54) VEIN PROJECTOR CAPABLE OF IMAGE CALIBRATION AND METHOD OF IMAGE CALIBRATION

(71) Applicant: LinkSens Technology Inc., Jiangsu (CN)

(72) Inventor: Dahong Qian, Jiangsu (CN)

(21) Appl. No.: 14/315,287

(22) Filed: Jun. 25, 2014

(30) Foreign Application Priority Data
Jun. 26, 2013 (CN) 201310279383.6

(51) Int. Cl. G06T 7/00; H04N 5/33

(52) U.S. Cl. CPC G06T 7/0018; H04N 5/33
USPC 348/77

(57) ABSTRACT

A vein projector capable of image calibration is disclosed, which includes a first image capture module for capturing an infrared (IR) image of a vein, a control circuit, a camera lens for projecting an image, and a second image capture module for capturing the projected image. The control circuit is configured to generate an image for projection from the captured IR image. The image for projection is then projected by the camera lens, and the projected image is captured by the second image capture module. The first and second image capture modules are disposed substantially at the same position. The control circuit is further configured to compare the position of the captured IR image and the position of the captured projected image and to calibrate the generated image for projection. Also disclosed is a method of calibrating an image of a vein for projection.

### CROSS-REFERENCE TO RELATED APPLICATIONS

This application claims the priority of Chinese patent application number 201310279383.6, filed on Jun. 26, 2013, the entire contents of which are incorporated herein by reference.

### TECHNICAL FIELD

The present invention relates to auxiliary medical equipment, and particularly to a vein projector capable of image calibration. The present invention also relates to a method of image calibration for use in a vein projector.

### BACKGROUND

Venipuncture is a common method for administering a medication. However, as subcutaneous veins are hard to be identified by human eyes, the administration is generally a challenging operation for medical personnel, especially for doctors or nurses in their internship, who may need to make several attempts before successfully locating a vein, causing the patient to experience more pain.

There are a number of types of vein projectors that can be used to facilitate the vein location, for example, the one disclosed in Chinese Pat. Pub. No. CN 1621889 A, as shown in FIG. 1, which includes an infrared (IR) light source 1, an IR camera 2, an IR reflector 3, an imaging device 4, a blue-green light source 5, and a camera lens 6. The IR light source 1 illuminates the palm of a hand of a patient, such that the camera lens 6 captures an IR image of a vein in the hand and projects the IR image to the IR reflector 3. The IR reflector 3 then reflects the IR image to the IR camera 2 which identifies the IR image and transfers it to the imaging device 4. As the IR imaging device 4 is illuminated by the blue-green light source 5, it reproduces the IR image as a blue-green image, which is fed via the IR reflector 3 back to the camera lens 6 and projected thereby on the back of the patient's hand.

This conventional vein projector is associated with the following problems: 1) as an open-loop system, it is incapable of detecting or correcting, a positional deviation between a graphical representation of the vein in the projected image and the actual vein; and 2) there is no suitable wavelength range prescribed for the IR light source, which leads to unreliable performance of the device in vein location.

### SUMMARY OF THE INVENTION

Accordingly, it is an objective of the present invention to provide a vein projector which is capable of detecting a destination position of an image of a vein for projection and spontaneously calibrating it when it is deviated from the actual position of the vein. It is a further objective of the present invention to provide a method of image calibration for use in a vein projector.

The foregoing objectives are attained by a vein projector that includes: a substrate configured to accommodate a body portion; a near infrared (NIR) light source, disposed on the substrate and configured to emanate NIR light to illuminate the body portion; a first image capture module configured to capture an infrared (IR) image of a vein in the body portion generated by the illumination of the NIR light source; a control circuit disposed above the substrate and including a controller module, coupled to the first image capture module for receiving the captured IR image of the vein, an imaging module coupled to the controller module and configured to generate an image for projection from the captured IR image of the vein, and a projection light source configured to illuminate the imaging module; a camera lens configured to project the image for projection onto the body portion; and a second image capture module coupled to the controller module and configured to capture the image projected on the body portion and transfer the captured projected image to the controller module, wherein the first and second image capture modules are arranged substantially at the same position; and wherein the controller module is configured to compare the position of the captured IR image and the position of the captured projected image and to calibrate the image for projection generated by the imaging module.

Preferably, the first and second image capture modules are two stand-alone cameras arranged substantially at the same position alongside each other.

Alternatively and also preferably, the second image capture module is a camera, and the first image capture module consists of the camera and an IR filter lens.

Preferably, the NIR light source is a light-emitting diode (LED) light source having a wavelength in the range of 660-1100 nm.

Preferably, the projection light source is a blue-green light source.

Preferably, the imaging module is a liquid crystal on silicon (LCOS) projection imaging device.

The foregoing objectives are also attained by a method of calibrating an image of a vein for projection that includes the steps of: capturing an IR image of a vein in a body portion at a first position; generating the image for projection from the captured IR image of the vein and projecting the image for projection onto the body portion; capturing the image projected on the body portion at a second position which is substantially the same as the first position; and calibrating the image for projection based on the captured IR image and the captured projected image.

Preferably, the first position is coincident with the second position.

Alternatively and also preferably, the first position is closely adjacent to the second position.

Preferably, the calibration includes correcting the image for projection with a compensation amount.

From the above description, it can be understood that the vein projector of the present invention has the advantage of being capable of detecting a destination position of an image of a vein for projection and spontaneously calibrating the image of the vein when it is deviated from the actual position of the vein.

### BRIEF DESCRIPTION OF DRAWINGS

To further describe the present invention, reference is made to the following detailed description on preferred embodiments, taken in conjunction with the accompanying drawings, in which:

FIG. 1 shows a conventional vein projector.

FIG. 2 is a schematic of a vein projector in accordance with a first embodiment of the present invention.

FIG. 3 schematically illustrates a control circuit of the vein projector of FIG. 2.

FIG. 4 is a schematic of a vein projector in accordance with a second embodiment of the present invention.

FIG. 5 schematically illustrates a control circuit of the vein projector of FIG. 4.

FIG. 6 depicts a flow chart graphically illustrating a method of image calibration for use in a vein projector in accordance with the present invention.

FIG. 7 is a flow chart illustrating a specific example of the method of FIG. 6.

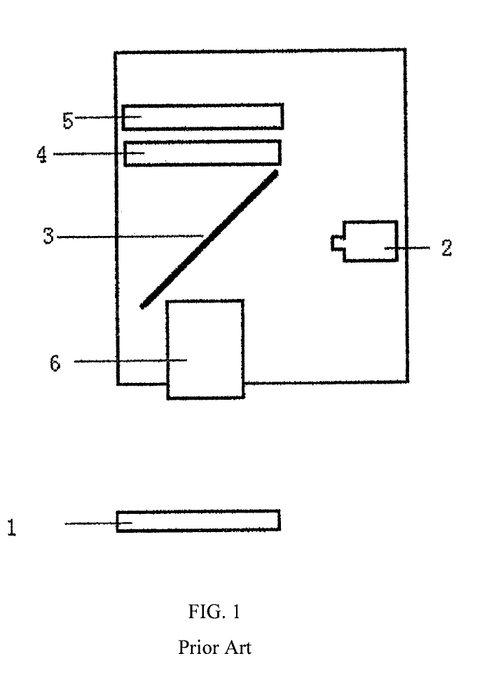

### DETAILED DESCRIPTION

When human body is illuminated with a light-emitting diode (LED) light source which produces near infrared (NIR) light having a wavelength in the range of 660 nm to 1100 nm, the NIR light emanating from the LED light source will travel through bones and muscle tissues easily and be substantially absorbed by hemoglobin in veins. This allows a Complementary Metal-Oxide-Semiconductor (CMOS) camera or an infrared Charge-Coupled Device (CCD) imager to capture an image of the veins.

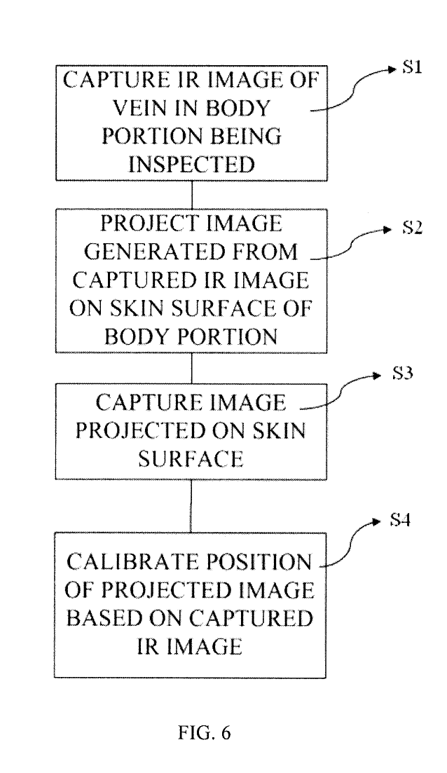

Based on this concept, the present invention provides a method of image calibration for use in a vein projector, as shown in FIG. 6, which is capable of projecting an image of a vein in a body portion directly onto the surface skin thereof. The method includes the following steps:

S1) capturing an IR image of the vein at a first position;

S2) generating an image for projection from the captured IR image and projecting it onto the body portion;

S3) capturing the image projected on the body portion at a second position which is substantially the same as the first position; and

S4) calibrating the image for projection based on the captured IR image and the captured projected image.

Steps S1) and S3) may be accomplished either by the same CMOS image sensor used in combination with a filter lens or not (i.e., the first and second positions are the same), or individually by respective CMOS image sensors arranged substantially at the same position. More detailed description of this will be given below with reference to the following two preferred embodiments.

### Embodiment 1

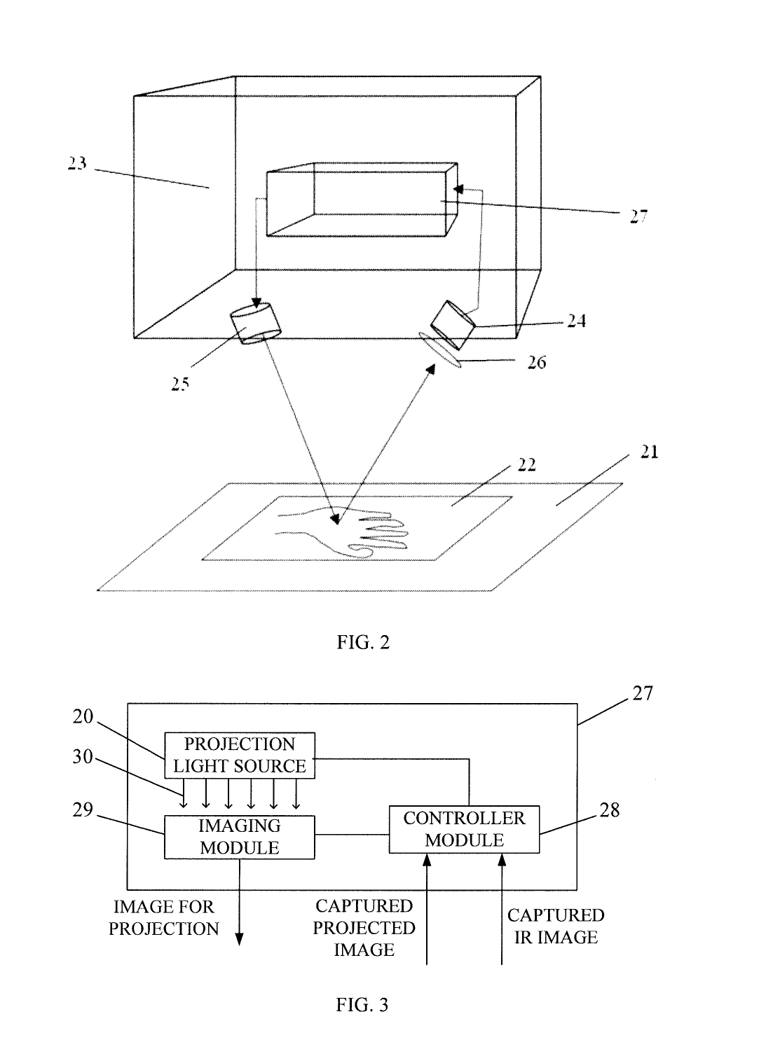

FIG. 2 depicts a vein projector capable of image calibration in accordance with this embodiment, which is able to project an image of a vein directly onto the skin surface of a body portion being inspected (e.g., the hand shown in FIG. 2) where the vein is located, and perform a calibration on the position of the projected image. Referring to FIG. 2, in conjunction with FIG. 6, the vein projector in this embodiment uses the same CMOS image sensor, which is used in combination with a filter lens or not, to carry out steps S1) and S3). As shown in FIG. 2, the vein projector includes a substrate 21 and a projector main body 23.

The projector main body 23 includes a near infrared (NIR) light source 22, which is disposed on the substrate 21 at a position corresponding to a position of the body portion. In this embodiment, the NIR light source 22 is a light-emitting diode (LED) light source having a wavelength in the range of 660 nm to 1100 nm.

The projector main body 23 further includes a first image capture module (i.e., consisting of a camera 24 and a filter lens 26) for capturing an IR image of the vein and a control circuit 27. As shown in FIG. 3, the control circuit 27 includes a controller module 28, an imaging module 29 and a projection light source 20. The controller module 28 has an input terminal connected to the first image capture module and an output terminal connected to the imaging module 29, such that it can control the imaging module 29 to generate an image for projection from the IR image of the vein captured by the first image capture module. The imaging module 29 may be a liquid crystal on silicon (LCOS) projection imaging device. The projection light source 20 is configured to emanate light 30 for illuminating the imaging module 29. Preferably, the projection light source 20 is a blue-green light source (i.e., the light 30 is blue-green light).

The projector main body 23 further includes a camera lens 25, and a second image capture module (i.e., the camera 24) coupled to the controller module 28. The camera lens 25 is configured to project the image for projection generated by the imaging module 29 on the skin surface of the body portion being inspected, whilst the second image capture module is configured to capture the image projected on the body portion and transfer the captured projected image to the controller module 28. Upon receiving the captured projected image, the controller module 28 compares its position with the position of the captured IR image and performs a calibration on the image for projection based on the comparison. In this embodiment, the first and second image capture modules are implemented as the same camera 24, which is arranged above the substrate 21 and coupled to the controller module 28. The camera 24 can be provided with a filter lens 26 mounted on its front end, which only allows light in a certain spectral band, for example, IR light, to pass. As such, the camera 24 functions as the first image capture module for capturing the IR image when the filter lens 26 is mounted thereon, and the camera 24 functions as the second image capture module, without the filter lens 26 mounted thereon, for capturing an ordinary image, such as the image projected on the body portion.

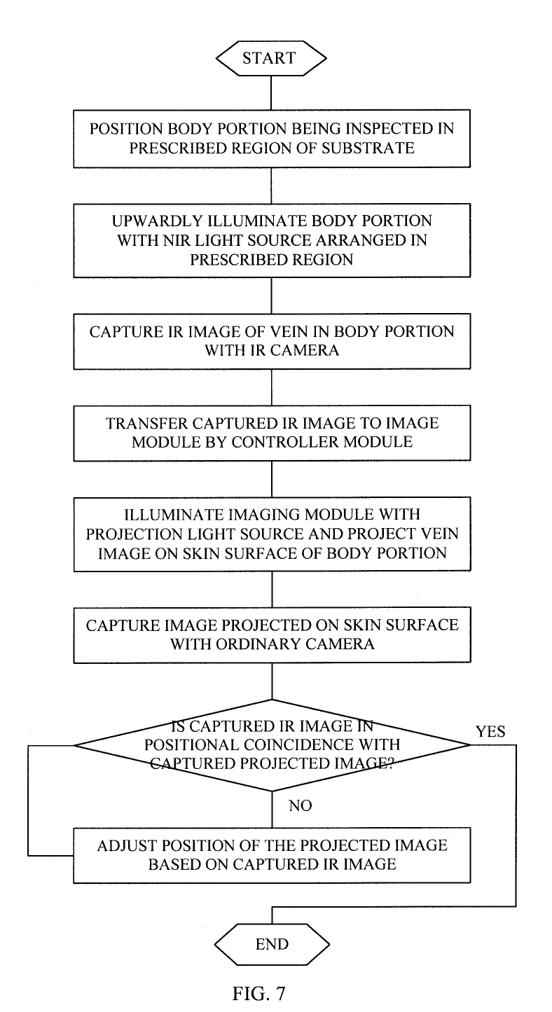

FIG. 7 depicts a flow chart illustrating an example of a method of image calibration in accordance with the present invention for use in the vein projector shown in FIG. 2.

The method includes the steps of:

1) positioning the body portion in a prescribed region of the substrate 21;

2) the NIR light source 22 arranged in the prescribed region upwardly illuminating the body portion;

3) the camera 24 (mounted with the filter lens 26) capturing an IR image of a vein in the body portion;

4) the controller module 28 transferring the IR image of the vein to the imaging module 29;

5) the projection light source 20 illuminating the imaging module 29 and the camera lens 25 projecting an image of the vein onto the body portion;

6) the camera 24 (with the filter lens 26 detached therefrom) capturing the image projected on the body portion; and

7) detecting whether the captured IR image is in positional coincidence with the captured projected image, and if not, adjusting the position of the image to be projected based on the position of the captured IR image such that they are in coincidence.

The adjustment may include, for example, if there is a positional deviation or local variance between the two captured images, then repeating the process of correcting the image to be projected by applying thereto a compensation amount, projecting the image to be projected and capturing the projected image, until a coincidence between the two images is achieved.

### Embodiment 2

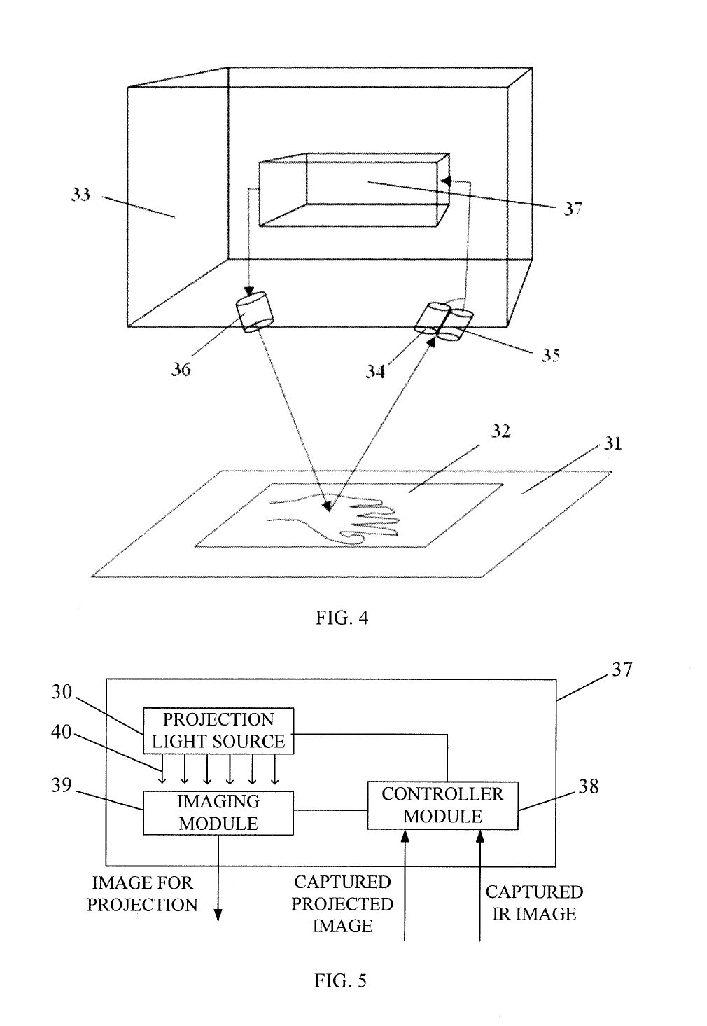

FIG. 4 depicts a vein projector capable of image calibration in accordance with this embodiment. The vein projector uses two independent CMOS image sensors arranged substantially at the same position to respectively carry out steps S1) and S3) of the method of FIG. 6. As shown in FIG. 4, the vein projector includes a substrate 31 and a projector main body 33.

The projector main body 33 includes an NIR light source 32, which is disposed on the substrate 31 at a position corresponding to a body portion being inspected (e.g., the hand shown in FIG. 4). In this embodiment, the NIR light source 32 is an LED light source having a wavelength in the range of 660 nm to 1100 nm.

The projector main body 33 further includes a first image capture module (i.e., a camera 35) for capturing an IR image of a vein in the body portion and a control circuit 37. As shown in FIG. 5, the control circuit 37 includes a controller module 38, an imaging module 39 and a projection light source 30. The controller module 38 has an input terminal connected to the first image capture module and an output terminal connected to the imaging module 39, such that it can control the imaging module 39 to generate an image for projection from the IR image of the vein captured by the first image capture module. The imaging module 39 may be a LCOS projection imaging device. The projection light source 30 is configured to emanate light 40 for illuminating the imaging module 39. Preferably, the projection light source 30 is a blue-green light source (i.e., the light 40 is blue-green light).

The projector main body 33 further includes a camera lens 36, and a second image capture module (i.e., another camera 34) coupled to the controller module 38. The camera lens 36 is configured to project the image for projection generated by the imaging module 39 on the skin surface of the body portion being inspected, whilst the second image capture module is configured to capture the image projected on the body portion and transfer the captured projected image to the controller module 38. Upon receiving the captured projected image, the controller module 38 compares its position with the position of the captured IR image and performs a calibration on the image for projection based on the comparison.

In this embodiment, the first and second image capture modules are implemented as a second camera 35 and a first camera 34, which are arranged substantially at the same position above the substrate 31 and both coupled to the controller module 38, in order to capture the IR image and the projected image, respectively. The first camera 34 may be a CMOS image sensor, and the second camera 35 may be consisted of a CMOS image sensor and an IR filter lens. As used herein, the phase “substantially at the same position” denotes that as the two stand-alone CMOS sensors are miniature cameras, both small in size and arranged in close proximity to each other (i.e., resembling two adjacent pixels of an image sensor array), they can be envisioned as being located at the same position.

FIG. 7 depicts a flow chart illustrating an example of a method of image calibration in accordance with the present invention for use in the vein projector shown in FIG. 4.

The method includes the steps of:

1) positioning the body portion in a prescribed region of the substrate 31;

2) the NIR light source 32 arranged in the prescribed region upwardly illuminating the body portion;

3) the second camera 35 as shown in FIG. 4 capturing an IR image of a vein in the body portion;

4) the controller module 38 transferring the IR image of the vein to the imaging module 39;

5) the projection light source 30 illuminating the imaging module 39 and the camera lens 36 projecting an image of the vein onto the body portion;

6) the first camera 34 as shown in FIG. 4 capturing the image projected on the body portion; and

7) detecting whether the captured IR image is in positional coincidence with the captured projected image, and if not, adjusting the position of the image to be projected based on the position of the captured IR image such that they are in coincidence.

The adjustment may include, for example, detecting whether there is a difference in position or shape between the two captured images using an image recognition technology, and if a difference is detected, then repeating the process of adjusting the image to be projected based on the captured IR image, projecting the image to be projected and capturing the projected image, until a coincidence between the two images is detected.

It is to be understood that the foregoing preferred embodiments are not intended to limit the invention in any way. Those skilled in the art can make various modifications and variations without departing from the spirit of the invention. Thus, it is intended that the present invention covers all such modifications and variations, provided they come within the true scope of the present invention.

What is claimed is:

1. A vein projector, comprising:
a substrate configured to accommodate a body portion;
a near infrared (NIR) light source, disposed on the substrate and configured to emanate an NIR light to illuminate the body portion;
a first image capture module configured to capture an infrared (IR) image of a vein in the body portion generated by the illumination of the NIR light source;
a control circuit disposed above the substrate and comprising:
a controller module, coupled to the first image capture module for receiving the captured IR image of the vein;
an imaging module, coupled to the controller module and configured to generate an image for projection from the captured IR image of the vein; and
a projection light source configured to illuminate the imaging module;
a camera lens configured to project the image for projection onto the body portion; and
a second image capture module, coupled to the controller module and configured to capture the image projected on the body portion and transfer the captured projected image to the controller module,
wherein the first and second image capture modules are arranged substantially at a same position; and
wherein the controller module is configured to compare a position of the captured IR image and a position of the captured projected image and to calibrate the image for projection generated by the imaging module.

2. The vein projector of claim 1, wherein the first and second image capture modules are two stand-alone cameras arranged substantially at the same position alongside each other.

3. The vein projector of claim 1, wherein the second image capture module is a camera, and the first image capture module consists of the camera and an IR filter lens.

4. The vein projector of claim 1, wherein the NIR light source is a light-emitting diode light source having a wavelength of 660 nm to 1100 nm.

5. The vein projector of claim 1, wherein the projection light source is a blue-green light source.

6. The vein projector of claim 1, wherein the imaging module is a liquid crystal on silicon projection imaging device.

7. A method of calibrating an image of a vein for projection, comprising the steps of:
capturing, at a first position, an IR image of a vein in a body portion;
generating an image for projection from the captured IR image of the vein and projecting the image for projection onto the body portion;
capturing, at a second position, the image projected on the body portion, wherein the second position is substantially the same as the first position; and
calibrating the image for projection based on the captured IR image and the captured projected image.

8. The method of claim 7, wherein the first position is coincident with the second position.

9. The method of claim 7, wherein the first position is closely adjacent to the second position.

10. The method of claim 7, wherein the calibration includes correcting the image for projection with a compensation amount.
